(19) United States
(12) Patent Application Publication
(10) Pub. No.: US 2025/0054572 A1
(43) Pub. Date: Feb. 13, 2025

(54) PROTEIN DOCKING METHOD, ELECTRONIC DEVICE, AND STORAGE MEDIUM

(71) Applicant: BEIJING BAIDU NETCOM SCIENCE TECHNOLOGY CO., LTD., Beijing (CN)

(72) Inventors: Jie Gao, Beijing (CN); Jing Hu, Beijing (CN); Xiaomin Fang, Beijing (CN); Xiaonan Zhang, Beijing (CN)

(73) Assignee: BEIJING BAIDU NETCOM SCIENCE TECHNOLOGY CO., LTD., Beijing (CN)

(21) Appl. No.: 18/930,048

(22) Filed: Oct. 29, 2024

(30) Foreign Application Priority Data
Nov. 1, 2023 (CN) 202311443053.6

(51) Int. Cl. G16B 15/30

(52) U.S. Cl. CPC G16B 15/30

(57) ABSTRACT

A method for protein docking includes docking a first protein and a second protein according to at least two molecular docking methods to generate a complex conformation in each round of iteration; recognizing that an iteration end condition is not satisfied, continuing a next round of iteration until the iteration end condition is satisfied, and obtaining a final complex conformation.

### CROSS REFERENCE TO RELATED APPLICATION

This application is based on and claims priority to Chinese patent application No. 2023114430536, filed on Nov. 1, 2023, the entire content of which is hereby introduced into this application as a reference.

### TECHNICAL FIELD

The present disclosure relates to the technical field of artificial intelligence, and in particular to the technical field of biocomputing, molecular docking, and deep learning, and in particular to a method for protein docking, and an electronic device, a storage medium and a computer program product.

### BACKGROUND

A protein docking refers to a process of forming a complex (also called a composite) by interaction between two or more protein molecules, which plays an important role in the biological field. The protein docking may include, for example, antigen-antibody docking, peptide-protein docking, etc. However, a method for protein docking in the related arts suffers from a low docking accuracy.

### SUMMARY

According to a first aspect of the present disclosure, a method for protein docking is provided. The method includes: docking a first protein and a second protein according to at least two molecular docking methods to generate a complex conformation in each round of iteration; recognizing that an iteration end condition is not satisfied, continuing a next round of iteration until the iteration end condition is satisfied, and obtaining a final complex conformation.

According to a second aspect of the present disclosure, an electronic device is provided. The electronic device includes: at least one processor; and a memory communicatively coupled to the at least one processor; in which, the at least one processor is configured to dock a first protein and a second protein according to at least two molecular docking methods to generate a complex conformation in each round of iteration; recognize that an iteration end condition is not satisfied, continue a next round of iteration until the iteration end condition is satisfied, and obtain a final complex conformation.

According to a fourth aspect of the present disclosure, a non-transitory computer readable storage medium, having computer instructions stored thereon, which causes a computer to perform a method for protein docking according to the first aspect.

It should be appreciated that the description in this section is not intended to identify key or important features of embodiments of the present disclosure, and is also intended to limit the scope of the present disclosure. Other features of the present disclosure will be readily understood by the following specification.

### BRIEF DESCRIPTION OF THE DRAWINGS

The accompanying drawings are used for a better understanding of the solution and do not constitute a limitation of the present disclosure.

FIG. 1 is a flowchart illustrating a method for protein docking according to an embodiment of the present disclosure.

FIG. 2 is a flowchart illustrating a method for protein docking according to another embodiment of the present disclosure.

FIG. 3 is a flowchart illustrating a method for protein docking according to another embodiment of the present disclosure.

FIG. 4 is a flowchart illustrating a method for protein docking according to another embodiment of the present disclosure.

FIG. 5 is a structure diagram illustrating an apparatus for protein docking according to an embodiments of the present disclosure.

FIG. 6 is a block diagram illustrating an electronic device according to an embodiment of the present disclosure.

### DETAILED DESCRIPTION

Exemplary embodiments of the present disclosure are described hereinafter in conjunction with the accompanying drawings, which include various details of the embodiments of the present disclosure in order to aid in understanding, and which should be considered exemplary only. Accordingly, one of ordinary skill in the art should recognize that various changes and modifications can be made to the embodiments described herein without departing from the scope and spirit of the present disclosure. Similarly, descriptions of well-known features and structures are omitted from the following description for the sake of clarity and brevity.

An AI (Artificial Intelligence) is a technical science that studies and develops theories, methods, technologies and application systems for simulating, extending and expanding human intelligence. At present, AI technology has advantages of high automation, high accuracy and low cost, and has been widely used.

Biocomputing refers to a computational model using a biomacromolecule as “data”, which is mainly categorized into three types: a protein computing, a ribonucleic acid (RNA) computing, and a deoxyribonucleic acid (DNA) computing, or refers to a subfield of computer science and computer engineering that uses bioengineering and biology to build computers, but similar to bioinformatics, which is an interdisciplinary science of using computers to store and process biological data.

Molecular docking is a method of drug design based on a characterization of a receptor and an interaction between the receptor and a drug molecule. The molecular docking is a method of theoretical simulation which focuses on the study of an interaction between molecules (for example, ligand and receptor) and predicts binding modes and affinities of the molecules. In recent years, the method of molecular docking has become an important technique in the field of computer-aided drug research.

Deep learning (DL) is a new research direction in the field of machine learning (ML), which is a science of learning intrinsic laws and representation levels of sample data, enabling a machine to be able to analyze and learn like a human being, and to recognize data such as text, images, and sounds. The deep learning is widely used in speech and image recognition.

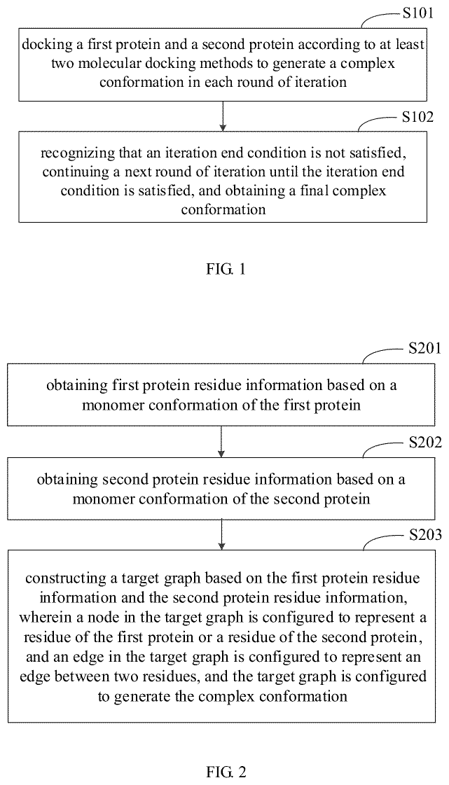

Referring to FIG. 1, FIG. 1 is a flowchart illustrating a method for protein docking according to an embodiment of the present disclosure. As shown in FIG. 1, the method includes the following steps S101 to S102.

At step S101, a first protein and a second protein are docked according to at least two molecular docking methods to generate a complex conformation in each round of iteration.

It is noted that an execution subject of the method for protein docking in an embodiment of the present disclosure may be a hardware device having data information processing capability and/or necessary software required to drive the hardware device to operate. Alternatively, the execution subject may include a workstation, a server, a computer, a user terminal, and other intelligent devices. The user terminal may include, but is not limited to, a cell phone, a computer, an intelligent voice interaction device, an intelligent home appliance, a vehicle-mounted terminal, and the like.

It is noted that the at least two molecular docking methods are not limited, for example, may include rigid docking, flexible docking, semi-flexible docking, and the like. During the rigid docking, neither a monomer conformation of the first protein nor a monomer conformation of the second protein appears to be changed. During the flexible docking, at least one of the monomer conformation of the first protein and the monomer conformation of the second protein appears to change.

It is noted that an order in which the at least two molecular docking methods are performed is not limited, for example, in a case that the at least two molecular docking methods includes the rigid docking and the flexible docking, the first protein and the second protein may be docked in an order of the rigid docking first and the flexible docking later, to generate the complex conformation, or, the first protein and the second protein may be docked in an order of the flexible docking first and the rigid docking later, to generate the complex conformation.

It is appreciated that each molecular docking method may generate one complex conformation. In a case that the complex conformation does not currently exist, the complex conformation may be generated according to one molecular docking method. In a case that the complex conformation currently exists, the complex conformation may be re-generated, i.e., the complex conformation may be updated, according to one molecular docking method.

For example, during each round of iteration, the first protein and the second protein are docked according to a rigid docking method to generate the composite conformation, and the composite conformation is updated according to a flexible docking method.

For example, during each round of iteration, the first protein and the second protein are docked according to the flexible docking method to generate the composite conformation, and the composite conformation is updated according to the rigid docking method.

At S102, it is recognized that an iteration end condition is not satisfied, a next round of iteration is continued until the iteration end condition is satisfied, and obtaining a final complex conformation.

It is noted that the iteration end condition is not limited, for example, the iteration end condition may include the number of rounds of iteration reaching a preset threshold, a parameter of the complex conformation being in a preset range, the first protein and the second protein being docked successfully, and the like. The parameter of the complex conformation is used to characterize a docking accuracy.

In an embodiment, obtaining the final complex conformation includes taking the last obtained complex conformation as the final complex conformation.

In an embodiment, obtaining the final complex conformation includes the final complex conformation selected from a plurality of complex conformations generated by the plurality of rounds of iteration.

It is noted that the method for protein docking provided in the present disclosure is applicable for at least one of following scenarios.

Scenario 1, antigen-antibody docking: the present disclosure may be configured to predict an antigen-antibody complex conformation, thus assisting in an antibody design.

Scenario 2, peptide-protein docking: the present disclosure may be configured to predict a peptide protein complex conformation, thus assisting in peptide drug design.

Scenario 3, disease mechanism research: occurrence and development of many diseases are related to abnormal interactions between the proteins, and the protein docking may help researchers to understand a molecular mechanism of the abnormal interactions, and thus provide new ideas for diagnosis and treatment of the diseases.

Scenario 4, protein function research: the protein docking may reveal the interactions between proteins and help scientists understand functions and regulatory mechanisms of the proteins, which are important for revealing biological processes such as cell signaling and gene regulation.

Scenario 5, structural biology: in protein structural biology, the protein docking may be configured to predict the binding mode of the protein complex, thus helping researchers to explain a structure and a function of the protein complex.

Scenario 6, disease mechanism research: the occurrence and development of many diseases are related to abnormal interactions between the proteins, and the protein docking may help researchers to understand the molecular mechanisms of the abnormal interactions, and thus provide new ideas for the diagnosis and treatment of the diseases.

Scenario 7, protein interaction network analysis: a protein interaction network may be constructed by predicting the interactions between proteins, which may help researchers understand interconnections and regulatory networks of proteins in a cell.

Scenario 8, protein engineering: in the field of biotechnology, the protein docking may be configured to design a new protein construct, such as fusion protein, antibody, enzyme, and the like, in order to realize a specific function.

In the method for protein docking provided in the present disclosure, the first protein and the second protein are docked according to the at least two molecular docking methods to generate the complex conformation in each round of iteration, it is recognized that the iteration end condition is not satisfied, the next round of iteration is continued until the iteration end condition is satisfied, and the final complex conformation is obtained. Therefore, the protein docking may be realized by considering the at least two molecular docking methods integration, and a plurality of rounds of protein docking can be performed, which may realize a plurality of rounds of iteration for the composite conformation, and an accuracy of the protein docking may be improved.

On the basis of any one of the above embodiments, a target graph may be constructed, and the target graph is used to generate the complex conformation.

Regarding the construction of the target graph, it may be further understood in connection with FIG. 2. FIG. 2 is a flowchart illustrating a method for protein docking according to another embodiment of the present disclosure. As shown in FIG. 2, the method includes the following steps S201 to S203.

At S201, first protein residue information is obtained based on a monomer conformation of the first protein.

At S202, second protein residue information is obtained based on a monomer conformation of the second protein.

It is noted that the residue information is not limited, for example, the residue information may include a class of a residue, a position of the residue (for example, a position where a-carbon atom of the residue is). The a-carbon atom refers to a first carbon atom of the residue.

It is appreciated that the monomer conformation of the protein carries information of the residue of the protein, and the information of the residue of the protein may be extracted from the monomer conformation of the protein.

For example, the information of the residue of the first protein may be extracted from the monomer conformation of the first protein.

At S203, a target graph is constructed based on the first protein residue information and the second protein residue information, in which a node in the target graph is configured to represent a residue of the first protein or a residue of the second protein, and an edge in the target graph is configured to represent an edge between two residues, and the target graph is configured to generate the complex conformation.

It is noted that the target graph is a heterogeneous graph, and the target graph includes two types of nodes, a first type of node is configured to represent the residue of the first protein, and a second type of node is configured to represent the residue of the second protein. Nodes in the target graph correspond one-to-one with the residues. The edge in the target graph is used to represent the edge between the residues corresponding to the two nodes connected by the edge.

It is appreciated that the number of nodes in the target graph is a sum of a number of residues of the first protein and a number of residues of the second protein.

It is appreciated that the edge may or may not exist between any two nodes in the target graph.

In embodiments of the present disclosure, constructing the target graph based on the first protein residue information and the second protein residue information, includes the following possible implementations.

Implementation 1, the target graph is generated by constructing respective nodes corresponding one-to-one with the residues, in which the residues include the residue of the first protein and the residue of the second protein.

Thus, the respective nodes corresponding one-to-one with the residues may be constructed in the method to realize the construction of the respective nodes.

Implementation 2, distances between a feature of a first residue and features of respective second residues are determined based on information of the first residue and information of the second residues, in which the first residue and the second residues belong to a target protein, and the target protein is the first protein or the second protein; a plurality of second residues are sorted in ascending order according to the distances and determining first N second residues after sorting as target residues, in which N is a positive integer; and connecting edges are added between a node corresponding to the first residue and respective node corresponding to the target residues to generate the target graph.

Thus, in the method, the distances between the feature of the first residue and the features of respective second residues are determined based on the information of the first residue and the information of the second residues, and the N second residues with the closest distances may be used as the target residues, and the connecting edges between the node corresponding to the first residue and respective node corresponding to the target residues is added. Thus, the construction of edges between the residues of the same protein is realized.

It is noted that the first residue and the second residue belong to the target protein, i.e., the first residue and the second residue belong to the same protein, for example, the first residue and the second residue belong to the first protein, or, the first residue and the second residue belong to the second protein.

It is noted that N is not limited, for example, N may be 10.

In an embodiment, determining the distances between the feature of the first residue and the features of respective second residues based on the information of the first residue and the information of the second residues may include performing feature extraction on the information of the first residue to obtain the feature of the first residue, performing feature extraction on the information of the second residue to obtain the feature of the second residue, and acquiring the distances between the feature of the first residue and the feature of the second residue.

In an embodiment, the information of the first residue and the information of the second residue may be input into a KNN (K-nearest neighbor) model, and the target residue is output from the KNN model.

It is noted that the distance is not limited, for example, the distance may include a Euclidean distance, a Manhattan distance, and the like.

Implementation 3, the residue of the first protein is taken as a third residue; the residue of the second protein is taken as a fourth residue; a connecting edge is added between a node corresponding to the third residue and a node corresponding to the fourth residue to generate the target graph.

Thus, in the method, the connecting edge is added between the node corresponding to the third residue and the node corresponding to the fourth residue. Thus, the construction of edges between residues of different proteins may be realized.

It is noted that the third residue is any residue of the first protein and the fourth residue is any residue of the second protein.

Implementation 4, a feature of the node is determined based on the information of the residue corresponding to the node.

It is noted that the feature of the node is not limited, for example, the feature of the node may include a class of the residue corresponding to the node, a position of the residue corresponding to the node, and the like.

Implementation 5, the feature of the edge is determined based on the information of the two residues corresponding to the edge.

It is noted that the feature of the edge is not limited, for example, the feature of the edge may include a length of the edge, an angular difference of the two residues corresponding to the edge, and the like.

In an embodiment, determining the feature of the edge based on the information of the two residues corresponding to the edge includes determining a distance between the two residues corresponding to the edge, based on the position of the two residues corresponding to the edge, as a length of the edge.

In an embodiment, determining the feature of the edge based on the information of the two residues corresponding to the edge includes obtaining the difference in the angles of the two residues corresponding to the edge as the angular difference of the two residues corresponding to the edge.

On the basis of any one of the above embodiments, the method further includes, updating the first protein residue information and the second protein residue information based on a most recently obtained complex conformation; returning to perform a step of constructing the target graph based on the first protein residue information and the second protein residue information to update the target graph. Therefore, the residue information can be updated using the latest complex conformation, and the target graph may be reconstructed using the updated residue information, and real-time updating of the target graph may be realized during the process of docking the proteins.

It is appreciated that the complex conformation carries the first protein residue information and the second protein residue information, and the first protein residue information and the second protein residue information may be extracted from the most recently obtained complex conformation, and previous first protein residue information is replaced by the extracted first protein residue information, previous second protein residue information is replaced by the extracted second protein residue information.

It is appreciated that updating the target graph may include deleting or adding the edge between nodes, which is not limited herein.

In some embodiments, after updating the information of the residues of the first protein and the information of the residues of the second protein, the method further includes updating a feature of the node and a feature of the edge in the target graph based on the first protein residue information and the second protein residue information, in which the feature of the node is determined based on information of the residue corresponding to the node, and the feature of the edge is determined based on information of the two residues corresponding to the edge. Therefore, the feature of the node and the feature of the edge in the target graph may be updated using the updated residue information, and real-time updating of the feature of the node and the feature of the edge may be realized in the docking process.

The method for protein docking provided in the present disclosure, the first protein residue information is obtained based on the monomer conformation of the first protein, the second protein residue information is obtained based on the monomer conformation of the second protein, and the target graph is constructed based on the first protein residue information and the second protein residue information to generate the complex conformation.

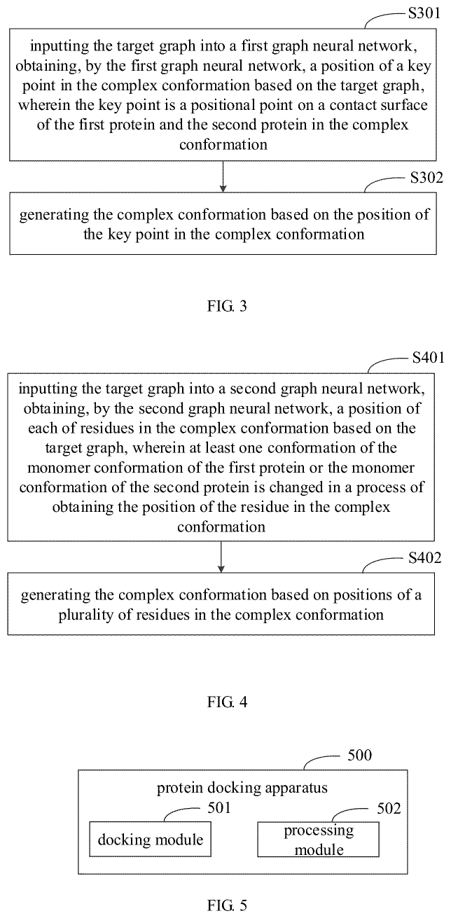

In the above embodiments, the at least two molecular docking methods include rigid docking, and the step S102 of docking the first protein and the second protein according to the at least two molecular docking methods to generate the composite conformation can be further understood in combination to FIG. 3. FIG. 3 is a flowchart illustrating a method for protein docking according to another embodiment of the present disclosure. As shown in FIG. 3, the method includes the following steps S301 to S302.

At S301, the target graph is input into a first graph neural network, a position of a key point in the complex conformation is obtained, by the first graph neural network, based on the target graph, in which the key point is a positional point on a contact surface of the first protein and the second protein in the complex conformation.

It is noted that during the process of obtaining the position of the key point in the complex conformation, the monomer conformation of the first protein, and the monomer conformation of the second protein do not change.

It is noted that the first graph neural network is not limited, for example, the first graph neural network may include GCN (graph convolutional network), GRN (graph recurrent network), GAT (graph attention network), GAT (graph attention network), and the like.

It is noted in that the number of the key point is not limited.

In an embodiment, obtaining the position of the key point in the complex conformation by the first graph neural network based on the target graph includes updating the feature of the node and the feature of the edge in the target graph by the first graph neural network, and obtaining the position of the key point in the complex conformation by the first graph neural network based on the feature of the node and the feature of the edge. Thus, acquisition of the position of the key point in the complex conformation may be realized.

It is noted that updating, by the first graph neural network, the feature of the node and the feature of the edge in the target graph may be realized by adopting any method for updating a feature of a graph neural network in the related art, and will not be limited herein.

In an embodiment, obtaining, by the first graph neural network, the position of the key point in the complex conformation based on the target graph, includes obtaining, by the first graph neural network, a position of each of residues in the complex conformation based on the target graph, obtaining a distance between the third residue and the fourth residue based on the position of the third residue in the complex conformation and the position of the fourth residue in the complex conformation, and in response to the distance between the third residue and the fourth residue being less than a preset threshold, a position of a midpoint of an edge between the third residue and the fourth residue in the complex conformation is taken as the position of the key point in the complex conformation. In this embodiment, in response to the distance between the third residue and the fourth residue being less than the preset threshold, the midpoint of the edge between the third residue and the fourth residue is used as the key point.

At S302, the complex conformation is generated based on the position of the key point in the complex conformation.

In an embodiment, generating the complex conformation based on the position of the key point in the complex conformation, includes determining a receptor and a ligand from the first protein and the second protein; obtaining a rotation translation matrix based on a position of the key point in a monomer conformation of the ligand, and the position of the key point in the complex conformation; performing an overall spatial transformation on the monomer conformation of the ligand based on the rotation translation matrix; generating the complex conformation based on a transformed monomer conformation of the ligand and a monomer conformation of the receptor. Therefore, the rotation translation matrix may be obtained based on the position of the key point in the complex conformation, such that the overall spatial transformation of the monomer conformation of the ligand is performed, and the complex conformation is generated based on a combination of the transformed monomer conformation of the ligand and the monomer conformation of the receptor.

It is noted that performing the overall spatial transformation on the monomer conformation of the ligand refers to performing an overall rotation and an overall translation on the monomer conformation of the ligand.

In some embodiments, determining the receptor and the ligand from the first protein and the second protein includes using the first protein as the receptor and the second protein as the ligand, or, using the second protein as the receptor and the first protein as the ligand.

In some embodiments, obtaining the rotation translation matrix based on the position of the key point in the monomer conformation of the ligand, and the position of the key point in the complex conformation includes generating a first matrix based on positions of a plurality of key points in the monomer conformation of the ligand, and generating a second matrix based on the positions of the plurality of key points in the complex conformation, the second matrix being a product of the rotational translation matrix and the first matrix, and the rotation translation matrix is obtained by performing matrix decomposition on the second matrix based on the first matrix. It should be noted that the matrix decomposition may be realized using any one of matrix decomposition methods in related art, which may include, for example, SVD (singular value decomposition).

In some embodiments, obtaining the rotation translation matrix based on the position of the key point in the monomer conformation of the ligand, and the position of the key point in the complex conformation includes using elements within the rotation translation matrix as unknown values, constructing equations based on the position of the key point in the monomer conformation of the ligand, and the position of the key point in the complex conformation, and the elements within the rotation translation matrix, solving the equations to obtain a solution of the equations, taking the solution as the elements within the rotation translation matrix to generate the rotation translation matrix.

In some embodiments, performing the overall spatial transformation on the monomer conformation of the ligand based on the rotation translation matrix includes performing a spatial transformation on the positions of the residues of the ligand in the monomer conformation based on the rotation translation matrix, and obtaining the transformed monomer conformation of the ligand based on the positions of the plurality of residues of the ligand in the monomer conformation.

In the method for protein docking proposed in the present disclosure, the at least two molecular docking methods include the rigid docking, the target graph is input into the first graph neural network, the position of the key point in the complex conformation is obtained, by the first graph neural network, based on the target graph, in which the key point is the positional point on the contact surface of the first protein and the second protein in the complex conformation, the complex conformation is generated based on the position of the key point in the complex conformation. Thus, the rigid docking of the proteins may be realized.

In the above embodiments, the at least two molecular docking methods include flexible docking, and the step S102 of docking the first protein and the second protein according to the at least two molecular docking methods to generate the composite conformation may be further understood in combination to FIG. 4. FIG. 4 is a flowchart illustrating a method for protein docking according to another embodiment of the present disclosure. As shown in FIG. 4, the method includes the following steps S401 to S402.

At S401, the target graph is input into a second graph neural network, a position of each of residues in the complex conformation is obtained, by the second graph neural network, based on the target graph, in which at least one conformation of the monomer conformation of the first protein or the monomer conformation of the second protein is changed in a process of obtaining the position of the residue in the complex conformation.

It is noted that the second graph neural network may be referred to with the related content of the first graph neural network, which will not be repeated herein.

It is noted that the residues include the residue of the first protein and the residue of the second protein.

In an embodiment, obtaining, by the second graph neural network, a position of each of residues in the complex conformation based on the target graph includes updating the features of the nodes and the features of the edges in the target graph by the second graph neural network, obtaining the position of each residue in the complex conformation by the second graph neural network based on the features of the nodes and the features of the edges. Thus, acquisition of the position of each residue in the complex conformation may be realized.

It is noted that updating, by the second graph neural network, the feature of the node and the feature of the edge in the target graph may be realized by adopting any method for updating features of a graph neural network in the related art, which will not be limited herein.

At S402, the complex conformation is generated based on positions of a plurality of residues in the complex conformation.

In embodiments of the present disclosure, generating the complex conformation based on the positions of the plurality of residues in the complex conformation includes generating the complex conformation based on the positions of residues of a plurality of first proteins in the complex conformation, and the positions of residues of a plurality of second proteins in the complex conformation.

The method fir protein docking provided in the present disclosure, the at least two molecular docking methods include flexible docking, the target graph is input into the second graph neural network, the position of each of residues in the complex conformation is obtaining, by the second graph neural network, based on the target graph, in which at least one of the monomer conformation of the first protein or the monomer conformation of the second protein is changed in a process of obtaining the position of the residue in the complex conformation, and generating the complex conformation based on the positions of the plurality of residues in the complex conformation. Thus, flexible docking of the proteins may be realized.

On the basis of any of the above embodiments, during each round of iteration, the first protein and the second protein may be docked in an order of the rigid docking first and the flexible docking later, to generate the complex conformation.

For example, the target graph may be constructed based on the first protein residue information and the second protein residue information. The target graph is input into the first graph neural network, the position of the key point in the complex conformation is obtained by the first graph neural network. A complex conformation A is generated based on the position of the key point in the complex conformation.

The first protein residue information and the second protein residue information are updated based on the complex conformation A, and it is return to perform the step of constructing the target graph based on the first protein residue information and the second protein residue information, to update the target graph. The feature of the node and the feature of the edge in the target graph may also be are updated based on the first protein residue information and the second protein residue information.

The target graph is input to the second graph neural network, the position of each of residues in the complex conformation is obtained, by the second graph neural network, based on the target graph. A complex conformation B is generated based on the positions of the plurality of residues in the complex conformation.

On the basis of any of the above embodiments, during each round of iteration, the first protein and the second protein may be docked in an order of the flexible docking first and the rigid docking later, to generate the complex conformation.

For example, the target graph is constructed based on the first protein residue information and the second protein residue information, and the target graph is input into the second graph neural network, the position of each of residues in the complex conformation is obtained by the second graph neural network. A complex conformation C is generated based on positions of a plurality of residues in the complex conformation.

The first protein residue information and the second protein residue information are updated based on the complex conformation C, and it is returned to perform the step of constructing the target graph based on the first protein residue information and the second protein residue information, to update the target graph. The feature of the node and the feature of the edge in the target graph may also be updated based on the first protein residue information and the second protein residue information.

The target graph is input to the first graph neural network, the position of the key point in the complex conformation is obtained, by the first graph neural network, based on the target graph, and a complex conformation D is generated based on the position of the key point in the complex conformation.

In the technical solution of the present disclosure, collection, storage, use, processing, transmission, provision, disclosure and others of user personal information involved comply with relevant laws and regulations, and is not contrary to public order and morals.

The present disclosure also provides an apparatus for protein docking according to embodiments of the present disclosure, for realizing the above described method of protein docking.

Referring to FIG. 5, FIG. 5 is a block diagram illustrating an apparatus for protein docking according to an embodiment of the present disclosure.

As shown in FIG. 5, the apparatus 500 for protein docking includes a docking module 501 and a processing module 502.

The docking module 501 is configured to dock a first protein and a second protein according to at least two molecular docking methods to generate a complex conformation in each round of iteration.

The processing module 502 is configured to recognize that an iteration end condition is not satisfied, continue a next round of iteration until the iteration end condition is satisfied, and obtain a final complex conformation.

In an embodiment of the present disclosure, the apparatus further includes: a building module, configured to: obtain first protein residue information based on a monomer conformation of the first protein; obtain second protein residue information based on a monomer conformation of the second protein; construct a target graph based on the first protein residue information and the second protein residue information, in which a node in the target graph is configured to represent a residue of the first protein or a residue of the second protein, and an edge in the target graph is configured to represent an edge between two residues, and the target graph is configured to generate the complex conformation.

In an embodiment of the present disclosure, the at least two molecular docking methods include the rigid docking, the docking module 501 is further configured to: input the target graph into a first graph neural network, obtain, by the first graph neural network, a position of a key point in the complex conformation based on the target graph, in which the key point is a positional point on a contact surface of the first protein and the second protein in the complex conformation; generate the complex conformation based on the position of the key point in the complex conformation.

In an embodiment of the present disclosure, the docking module 501 is further configured to: update, by the first graph neural network, a feature of the node and a feature of the edge in the target graph; obtain, by the first graph neural network, the position of the key point in the complex conformation based on the feature of the node and the feature of the edge.

In an embodiment of the present disclosure, the docking module 501 is further configured to: determine a receptor and a ligand from the first protein and the second protein; obtain a rotation translation matrix based on a position of the key point in a monomer conformation of the ligand, and the position of the key point in the complex conformation; perform an overall spatial transformation on the monomer conformation of the ligand based on the rotation translation matrix; generate the complex conformation based on a transformed monomer conformation of the ligand and a monomer conformation of the receptor.

In an embodiment of the present disclosure, the at least two molecular docking methods include flexible docking, the docking module 501 is further configured to: input the target graph into a second graph neural network, obtain, by the second graph neural network, a position of each of residues in the complex conformation based on the target graph, in which at least one conformation of the monomer conformation of the first protein or the monomer conformation of the second protein is changed in a process of obtaining the position of the residue in the complex conformation; generate the complex conformation based on positions of a plurality of residues in the complex conformation.

In an embodiment of the present disclosure, the building module, is further configured to: update the first protein residue information and the second protein residue information based on a most recently obtained complex conformation; return to perform a step of constructing the target graph based on the first protein residue information and the second protein residue information to update the target graph.

In an embodiment of the present disclosure, after updating the first protein residue information and the second protein residue information, the building module is further configured to: update a feature of the node and a feature of the edge in the target graph based on the first protein residue information and the second protein residue information, in which the feature of the node is determined based on information of the residue corresponding to the node, and the feature of the edge is determined based on information of the two residues corresponding to the edge.

In an embodiment of the present disclosure, the building module is further configured to: determine distances between a feature of a first residue and features of respective second residues based on information of the first residue and information of the second residues, in which the first residue and the second residues belong to a target protein, and the target protein is the first protein or the second protein; sort a plurality of second residues in ascending order according to the distances and determine first N second residues after sorting as target residues, in which N is a positive integer; add connecting edges between a node corresponding to the first residue and respective nodes corresponding to the target residues to generate the target graph.

In an embodiment of the present disclosure, the building module is further configured to: take the residue of the first protein as a third residue; take the residue of the second protein as a fourth residue; add a connecting edge between a node corresponding to the third residue and a node corresponding to the fourth residue to generate the target graph.

The apparatus for protein docking provided in the present disclosure, the first protein and the second protein are docked according to the at least two molecular docking methods to generate the complex conformation in each round of iteration, it is recognized that the iteration end condition is not satisfied, the next round of iteration is continued until the iteration end condition is satisfied, and the final complex conformation is obtained. Therefore, the protein docking may be realized by considering the at least two molecular docking methods integration, and a plurality of rounds of protein docking can be performed, which may realize a plurality of rounds of iteration for the composite conformation, and an accuracy of the protein docking may be improved.

According to embodiments of the present disclosure, which also provide an electronic device, a readable storage medium, and a computer program product.

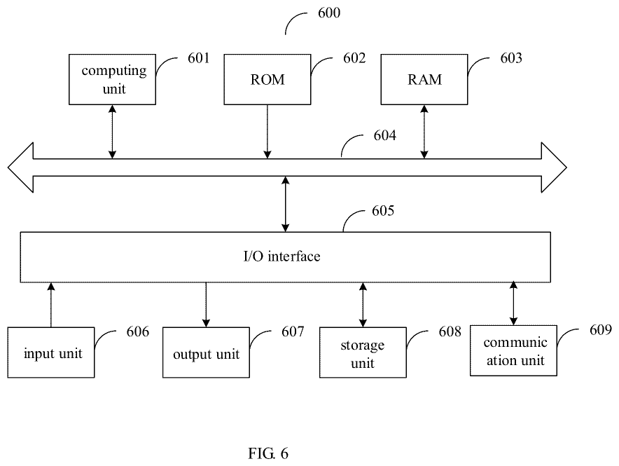

Referring to FIG. 6, which is a block diagram illustrating an electronic device according to an embodiment of the present disclosure. The electronic device is intended to represent various types of digital computers, such as laptop computers, desktop computers, workstations, personal digital assistants, servers, blade servers, mainframe computers, and other suitable computers. The electronic device may also represent various types of mobile apparatuses, such as personal digital assistants, cellular phones, smart phones, wearable non-intrusive flexible loads aggregation characteristic identification devices, and other similar computing devices. The components shown herein, their connections and relations, and their functions are merely examples, and are not intended to limit the implementation of the disclosure described and/or required herein.

As shown in FIG. 6, the device 600 includes a computing unit 601, configured to execute various appropriate actions and processes according to a computer program stored in a read-only memory (ROM) 602 or loaded from a storage unit 608 to a random access memory (RAM) 603. In the RAM 603, various programs and data required for the device 600 may be stored. The computing unit 601, the ROM 602 and the RAM 603 may be connected with each other by a bus 604. An input/output (I/O) interface 605 is also connected to the bus 604.

The device 600 are connected to an I/O interface 605, and includes: an input unit 606, for example, a keyboard, a mouse; an output unit 607, for example, various types of displays, speakers; a storage unit 608, for example, a magnetic disk, an optical disk; and a communication unit 609, for example, a network card, a modem, a wireless transceiver. The communication unit 609 allows the device 600 to exchange information/data through a computer network such as internet and/or various types of telecommunication networks and other devices.

The computing unit 601 may be various types of general and/or dedicated processing components with processing and computing ability. Some examples of a computing unit 601 include but not limited to a central processing unit (CPU), a graphics processing unit (GPU), various dedicated artificial intelligence (AI) computing chips, various computing units running a machine learning model algorithm, a digital signal processor (DSP), and any appropriate processor, controller, microcontroller, etc. The computing unit 601 executes various methods and processes as described above, for example, a method for protein docking. For example, in some embodiments, the method for protein docking may be further implemented as a computer software program, which is physically contained in a machine readable medium, such as the storage unit 608. In some embodiments, a part or all of the computer program may be loaded and/or installed on the device 600 via the ROM 602 and/or a communication unit 609. When the computer program is loaded on the RAM 603 and executed by the computing unit 601, one or more steps in the method for protein docking as described above may be performed. Alternatively, in other embodiments, the computing unit 601 may be configured to the method for protein docking in other appropriate ways (for example, by virtue of a firmware).

Various implementation modes of systems and technologies described herein may be implemented in a digital electronic circuit system, an integrated circuit system, a field programmable gate array (FPGA), a dedicated application specific integrated circuit (ASIC), a system on a chip (SOC), a complex programmable logic device (CPLD), a computer hardware, a firmware, a software, and/or combinations thereof. The implementations may include: implemented in one or more computer programs. The one or more computer programs may be executed and/or interpreted on a programmable system including at least one programmable processor. The programmable processor may be a dedicated or general-purpose programmable processor, may receive data and instructions from a storage system, at least one input device, and at least one output device, and transmit the data and the instructions to the storage system, the at least one input device, and the at least one output device.

A computer code configured to execute a method in the present disclosure may be written with one or any combination of multiple programming languages. These programming languages may be provided to a processor or a controller of a general-purpose computer, a dedicated computer, or other programmable apparatuses for data processing so that the function/operation specified in the flowchart and/or block diagram may be performed when the program code is executed by the processor or controller. A computer code may be executed completely or partly on the machine, executed partly on the machine as an independent software package and executed partly or completely on the remote machine or server.

In the embodiment of the present disclosure, a machine-readable medium may be a tangible medium that may contain or store a program intended for use in or in conjunction with an instruction execution system, an apparatus or a device. The machine-readable medium may be a machine-readable signal medium or a machine-readable storage medium. The machine-readable medium may include but not limited to an electronic, magnetic, optical, electromagnetic, infrared, or semiconductor system, apparatus or device, or any appropriate combination thereof. A more specific example of a machine readable storage medium includes an electronic connector with one or more cables, a portable computer disk, a hardware, a random access memory (RAM), a read-only memory (ROM), an EPROM programmable read-only ROM (an EPROM or a flash memory), an optical fiber device, and a compact disc read-only memory (CD-ROM), an optical storage device, a magnetic storage device, or any appropriate combination of the above.

In order to provide interaction with the user, the systems and technologies described here may be implemented on a computer, and the computer has: a display apparatus for displaying information to the user (for example, a CRT (cathode ray tube) or a LCD (liquid crystal display) monitor); and a keyboard and a pointing apparatus (for example, a mouse or a trackball) through which the user may provide input to the computer. Other types of apparatuses may further be configured to provide interaction with the user; for example, the feedback provided to the user may be any form of sensory feedback (for example, visual feedback, auditory feedback, or tactile feedback); and input from the user may be received in any form (including an acoustic input, a voice input or a tactile input).

Systems and technologies described herein may be implemented in a computing system (for example, as a data server) including a background component, or a computing system (for example, an application server) including a middleware component, or a computing system including a front-end component (for example, a user computer with a graphical user interface or a web browser, and the user may interact with implementations of the systems and technologies described herein via the graphical user interface or the web browser), or in a computing system including any combination of the background component, the middleware component, or the front-end component. Components of the system may be interconnected by any form or medium of digital data communication (for example, a communication network). Examples of the communication network include a local area network (LAN), a wide area network (WAN), and an Internet.

The computer system may include a client and a server. The client and the server are generally far away from each other and generally interact with each other through a communication network. The relationship between the client and the server is generated by computer programs that run on the corresponding computer and have a client-server relationship with each other. A server may be a cloud server, or a server with a distributed system, or a server in combination with a blockchain.

According to embodiments of the present disclosure, the present disclosure further provides a computer program product, including a computer program, which when executed by a processor to perform the steps of the method for protein docking described in the above embodiments of the present disclosure.

It should be noted that various forms of processes shown above may be used to reorder, add, or delete steps. For example, steps described in the present disclosure may be executed in parallel, sequentially, or in a different order, as long as the desired result of the technical solution disclosed in the present disclosure may be achieved, which will not be limited herein.

The above implementations do not constitute a limitation of the protection scope of the disclosure. Those skilled in the art shall understand that various modifications, combinations and sub-combinations and substitutions may be made. Any modification, equivalent substitution and improvement, etc., made within the spirit and principle of the present disclosure shall be included within the protection scope of the present disclosure.

What is claimed is:

1. A method for protein docking, comprising:
docking a first protein and a second protein according to at least two molecular docking methods to generate a complex conformation in each round of iteration;
recognizing that an iteration end condition is not satisfied, continuing a next round of iteration until the iteration end condition is satisfied, and obtaining a final complex conformation.

2. The method according to claim 1, comprising:
obtaining first protein residue information based on a monomer conformation of the first protein;
obtaining second protein residue information based on a monomer conformation of the second protein;
constructing a target graph based on the first protein residue information and the second protein residue information, wherein a node in the target graph is configured to represent a residue of the first protein or a residue of the second protein, and an edge in the target graph is configured to represent an edge between two residues, and the target graph is configured to generate the complex conformation.

3. The method according to claim 2, wherein the at least two molecular docking methods comprise rigid docking, and docking the first protein and the second protein according to the at least two molecular docking methods to generate the complex conformation comprises:
inputting the target graph into a first graph neural network, and obtaining, by the first graph neural network, a position of a key point in the complex conformation based on the target graph, wherein the key point is a positional point on a contact surface of the first protein and the second protein in the complex conformation;
generating the complex conformation based on the position of the key point in the complex conformation.

4. The method according to claim 3, wherein obtaining, by the first graph neural network, the position of the key point in the complex conformation based on the target graph comprises:
updating, by the first graph neural network, a feature of the node and a feature of the edge in the target graph;
obtaining, by the first graph neural network, the position of the key point in the complex conformation based on the feature of the node and the feature of the edge.

5. The method according to claim 3, wherein generating the complex conformation based on the position of the key point in the complex conformation comprises:
determining a receptor and a ligand from the first protein and the second protein;
obtaining a rotation translation matrix based on a position of the key point in a monomer conformation of the ligand, and the position of the key point in the complex conformation;
performing an overall spatial transformation on the monomer conformation of the ligand based on the rotation translation matrix;
generating the complex conformation based on a transformed monomer conformation of the ligand and a monomer conformation of the receptor.

6. The method according to claim 2, wherein the at least two molecular docking methods comprise flexible docking, and docking the first protein and the second protein according to the at least two molecular docking methods to generate the complex conformation comprises:
inputting the target graph into a second graph neural network, obtaining, by the second graph neural network, a position of each of residues in the complex conformation based on the target graph, wherein at least one conformation of the monomer conformation of the first protein or the monomer conformation of the second protein is changed in a process of obtaining the position of the residue in the complex conformation;
generating the complex conformation based on positions of a plurality of residues in the complex conformation.

7. The method according to claim 2, comprising:
updating the first protein residue information and the second protein residue information based on a most recently obtained complex conformation;
returning to perform a step of constructing the target graph based on the first protein residue information and the second protein residue information to update the target graph.

8. The method according to claim 7, wherein, after updating the first protein residue information and the second protein residue information, the method comprises:
updating a feature of the node and a feature of the edge in the target graph based on the first protein residue information and the second protein residue information, wherein the feature of the node is determined based on information of the residue corresponding to the node, and the feature of the edge is determined based on information of the two residues corresponding to the edge.

9. The method according to claim 2, wherein constructing the target graph based on the first protein residue information and the second protein residue information comprises:
determining distances between a feature of a first residue and features of respective second residues based on information of the first residue and information of the second residues, wherein the first residue and the second residues belong to a target protein, and the target protein is the first protein or the second protein;
sorting a plurality of second residues in ascending order according to the distances and determining first N second residues after sorting as target residues, wherein N is a positive integer;
adding connecting edges between a node corresponding to the first residue and respective node corresponding to the target residues to generate the target graph.

10. The method according to claim 2, wherein constructing the target graph based on the first protein residue information and the second protein residue information comprises:
taking the residue of the first protein as a third residue;
taking the residue of the second protein as a fourth residue;
adding a connecting edge between a node corresponding to the third residue and a node corresponding to the fourth residue to generate the target graph.

11. An electronic device, comprising:
at least one processor; and
a memory communicatively coupled to the at least one processor;
wherein the at least one processor is configured to:
dock a first protein and a second protein according to at least two molecular docking methods to generate a complex conformation in each round of iteration;
recognize that an iteration end condition is not satisfied, continue a next round of iteration until the iteration end condition is satisfied, and obtain a final complex conformation.

12. The electronic device according to claim 11, wherein the at least one processor is further configured to:
obtain first protein residue information based on a monomer conformation of the first protein;
obtain second protein residue information based on a monomer conformation of the second protein;
construct a target graph based on the first protein residue information and the second protein residue information, wherein a node in the target graph is configured to represent a residue of the first protein or a residue of the second protein, and an edge in the target graph is configured to represent an edge between two residues, and the target graph is configured to generate the complex conformation.

13. The electronic device according to claim 12, wherein the at least one processor is further configured to:
input the target graph into a first graph neural network, obtain, by the first graph neural network, a position of a key point in the complex conformation based on the target graph, wherein the key point is a positional point on a contact surface of the first protein and the second protein in the complex conformation;
generate the complex conformation based on the position of the key point in the complex conformation.

14. The electronic device according to claim 13, wherein the at least one processor is further configured to:
update, by the first graph neural network, a feature of the node and a feature of the edge in the target graph;
obtain, by the first graph neural network, the position of the key point in the complex conformation based on the feature of the node and the feature of the edge.

15. The electronic device according to claim 13, wherein the at least one processor is further configured to:
determine a receptor and a ligand from the first protein and the second protein;
obtain a rotation translation matrix based on a position of the key point in a monomer conformation of the ligand, and the position of the key point in the complex conformation;
perform an overall spatial transformation on the monomer conformation of the ligand based on the rotation translation matrix;
generate the complex conformation based on a transformed monomer conformation of the ligand and a monomer conformation of the receptor.

16. The electronic device according to claim 12, wherein the at least one processor is further configured to:
input the target graph into a second graph neural network, obtain, by the second graph neural network, a position of each of residues in the complex conformation based on the target graph, wherein at least one conformation of the monomer conformation of the first protein or the monomer conformation of the second protein is changed in a process of obtaining the position of the residue in the complex conformation;
generate the complex conformation based on positions of a plurality of residues in the complex conformation.

17. The electronic device according to claim 12, wherein the at least one processor is further configured to:
update the first protein residue information and the second protein residue information based on a most recently obtained complex conformation;
return to perform a step of constructing the target graph based on the first protein residue information and the second protein residue information to update the target graph.

18. The electronic device according to claim 17, wherein the at least one processor is further configured to:
update a feature of the node and a feature of the edge in the target graph based on the first protein residue information and the second protein residue information, wherein the feature of the node is determined based on information of the residue corresponding to the node, and the feature of the edge is determined based on information of the two residues corresponding to the edge.

19. The electronic device according to claim 12, wherein the at least one processor is further configured to perform one of:
determining distances between a feature of a first residue and features of respective second residues based on information of the first residue and information of the second residues, wherein the first residue and the second residues belong to a target protein, and the target protein is the first protein or the second protein, sorting a plurality of second residues in ascending order according to the distances and determine first N second residues after sorting as target residues, wherein N is a positive integer, and adding connecting edges between a node corresponding to the first residue and respective nodes corresponding to the target residues to generate the target graph; or
taking the residue of the first protein as a third residue, taking the residue of the second protein as a fourth residue, and adding a connecting edge between a node corresponding to the third residue and a node corresponding to the fourth residue to generate the target graph.

20. A non-transitory computer readable storage medium, having computer instructions stored thereon, which causes a computer to perform:
docking a first protein and a second protein according to at least two molecular docking methods to generate a complex conformation in each round of iteration;
recognizing that an iteration end condition is not satisfied, continuing a next round of iteration until the iteration end condition is satisfied, and obtaining a final complex conformation.
